(19) United States
(12) Patent Application Publication
(10) Pub. No.: US 2012/0253084 A1
(43) Pub. Date: Oct. 4, 2012

(54) VENT SCRUBBERS FOR USE IN PRODUCTION OF ETHANOL

(75) Inventors: Gerald Grusendorf, Rosharon, TX (US); Victor J. Johnston, Houston, TX (US); Fred Ronald Olsson, Corpus Christi, TX (US); Wayne D. Picard, Houston, TX (US); Nathan Powell, Waxahachie, TX (US); Samuel Roundy, League City, TX (US); Ismael Tejeda, Seabrook, TX (US); James Curtis, Dallas, TX (US)

(73) Assignee: CELANESE INTERNATIONAL CORPORATION, Dallas, TX (US)

(21) Appl. No.: 13/078,751

(22) Filed: Apr. 1, 2011

(51) Int. Cl. C07C 27/04; C07C 29/74

(52) U.S. Cl. 568/884; 568/913

(57) ABSTRACT

A process for producing ethanol comprising the steps of hydrogenating an acetic acid feed stream to form a crude ethanol product and separating at least a portion of the crude ethanol product to form an ethanol stream and at least one vent stream. The vent stream comprises non-condensable gases at least one volatile organic. The process further comprises the step of scrubbing the vent stream with at least two different solvents to recover the volatile organics. The vent stream may also comprise entrained ethanol, which may also be recovered.

### FIELD OF THE INVENTION

The present invention relates generally to processes for producing ethanol and, in particular, to vent scrubbers for recovering volatile organics and/or entrained ethanol. Embodiments of the present invention may use unreacted acid or byproducts, which are produced from hydrogenating acetic acid, as the scrubbing solvents for the vent scrubbers.

### BACKGROUND OF THE INVENTION

Ethanol for industrial use is conventionally produced from petrochemical feed stocks, such as oil, natural gas, or coal, from feed stock intermediates, such as syngas, or from starchy materials or cellulose materials, such as corn or sugar cane. Conventional methods for producing ethanol from petrochemical feed stocks, as well as from cellulose materials, include the acid-catalyzed hydration of ethylene, methanol homologation, direct alcohol synthesis, and Fischer-Tropsch synthesis. Instability in petrochemical feed stock prices contributes to fluctuations in the cost of conventionally produced ethanol, making the need for alternative sources of ethanol production all the greater when feed stock prices rise. Starchy materials, as well as cellulose material, are converted to ethanol by fermentation. However, fermentation is typically used for consumer production of ethanol for fuels or consumption. In addition, fermentation of starchy or cellulose materials competes with food sources and places restraints on the amount of ethanol that can be produced for industrial use.

Ethanol production via the reduction of alkanoic acids and/or other carbonyl group-containing compounds has been widely studied, and a variety of combinations of catalysts, supports, and operating conditions have been mentioned in the literature. During the reduction of alkanoic acid, e.g., acetic acid, other compounds are formed with ethanol or are formed in side reactions. These impurities limit the production of ethanol and typically require separation from the product ethanol, which is accomplished in a separation zone. Also, throughout the reaction and separation zones of conventional processes, volatile organics build up in the units thereof and must be vented with non-condensable gases. To control emissions and prevent loss of operating efficiencies, the volatile organics generally are recovered.

One typical method of recovering these volatile organics from the vent streams is to use a single stage scrubber that uses an aqueous solvent as a scrubbing solvent. US Pub. No. 2010/0196979 describes a wet scrubber to remove entrained ethanol and volatile organics from a vent stream of carbon dioxide produced via fermentation. This method, however, may not achieve the desired degree of recovery and a significant amount of volatile organics may be lost.

The need remains for an effective method for treating vent streams, which improves volatile organics recovery without adversely affecting the overall component balance of the separation zone.

### SUMMARY OF THE INVENTION

In one embodiment, the present invention is to process for producing ethanol. The process may comprise the step of providing at least one stream, e.g., vent stream, comprising non-condensable gases and at least one volatile organic. Preferably, the vent stream(s) are formed via the steps of hydrogenating an acetic acid feed stream to form a crude ethanol product; and separating at least a portion of the crude ethanol product to form an ethanol stream and the vent stream(s). The vent stream comprises non-condensable gases and at least one volatile organic. The process further comprises the step of scrubbing with a first scrubbing solvent at least a portion of the at least one vent stream to form a bottoms stream and an intermediate stream. Preferably, the first scrubbing solvent is acetic acid. The inventive process further comprises the step of scrubbing with a second scrubbing solvent at least a portion of the intermediate stream to form a scrubbed vent stream. The second scrubbing solvent is different from the first scrubbing solvent and, preferably, is water.

In another embodiment, the inventive process hydrogenates a first portion of an acetic acid feed stream to form a crude ethanol product and separates at least a portion of the crude ethanol product to form an ethanol stream, at least one vent stream comprising non-condensable gases and at least one volatile organic, and a water stream. The process scrubs at least a portion of the at least one vent stream with a second portion of the acetic acid feed stream to form an intermediate stream and scrubs at least a portion of the intermediate stream with at least a portion of the water stream to form a scrubbed vent stream.

In another embodiment, the process scrubs with a first scrubbing solvent at least a portion of the at least one vent stream to form a bottoms and a first intermediate stream, scrubs with a second scrubbing solvent different from the first scrubbing solvent at least a portion of the first intermediate stream to form a second intermediate stream, and scrubbs with a third scrubbing solvent different from the first scrubbing solvent at least a portion of the second intermediate stream to form a scrubbed vent stream.

### BRIEF DESCRIPTION OF THE DRAWINGS

FIG. 1 is a diagram of an ethanol production process in accordance with one embodiment of the present invention.

FIG. 2 is a schematic diagram of an exemplary ethanol production process in accordance with one embodiment of the present invention.

FIG. 3 shows a two-phase scrubbing unit in accordance with one embodiment of the present invention.

FIG. 4 shows a three-phase scrubbing unit in accordance with one embodiment of the present invention.

### DETAILED DESCRIPTION OF THE INVENTION

The present invention generally relates ethanol production, wherein one or more vent streams are treated using a multi-stage scrubbing unit. Preferably, the ethanol is produced via acetic acid hydrogenation, however, other production methods, e.g., ethyl acetate hydrogenolysis, are within the scope of the invention so long as these methods yield the one or more vent stream. The vent streams typically comprise non-condensable gases that are removed from the reactor, distillation columns, and/or storage tanks. Non-condensable gases may include nitrogen, methane, ethane, carbon dioxide, carbon monoxide, hydrogen, and mixtures thereof. In addition to the non-condensable gases, the vent streams may also comprise volatile organics and/or entrained ethanol. Volatile organics may include acids, esters, aldehydes, ketones, acetals, alcohols having three or more carbon atoms, and mixtures thereof. In one embodiment, the volatile organics may comprise acetic acid, ethyl acetate, acetaldehyde, acetal, and mixtures thereof. Embodiments of the present invention recover the volatile organics and entrained ethanol in a multi-stage scrubbing unit. A multi-stage scrubbing unit generally refers to a scrubber that uses at least two different scrubbing solvents. In one preferred embodiment the different scrubbing solvents may include an acidic solvent, such as acetic acid, and an aqueous solvent, such as water. Advantageously, multi-stage scrubbing units of the present invention may recover at least 95% of volatile organics and/or entrained ethanol from the vent streams, while reducing the emission of these components in the purged gases.

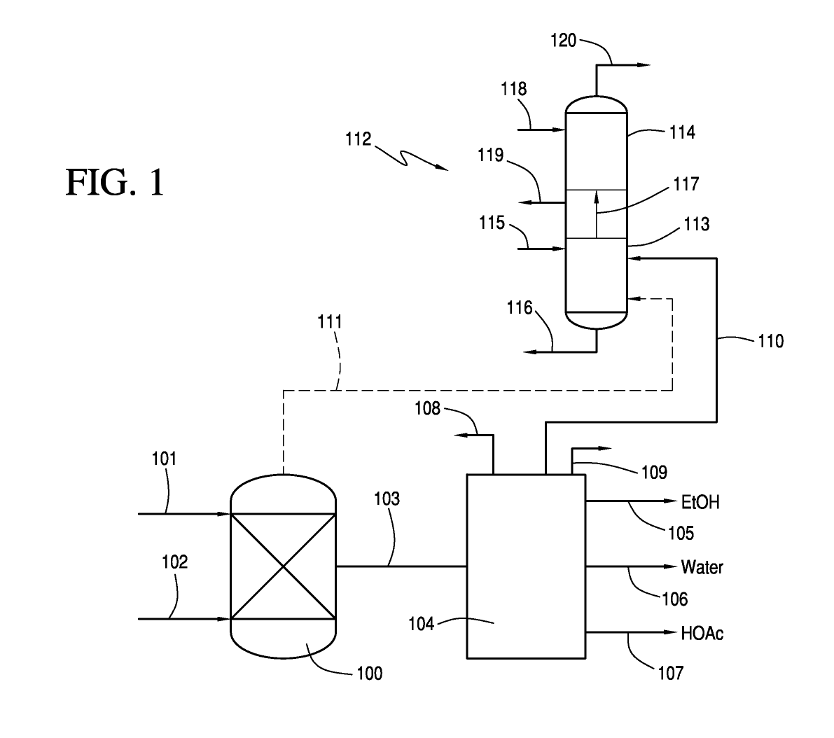

In one embodiment, the present invention is to a process for producing ethanol comprising the steps of hydrogenating an acetic acid feed stream to form a crude ethanol product and separating at least a portion of the crude ethanol product to form an ethanol product stream and at least one vent stream. As shown in FIG. 1, hydrogen and acetic acid are fed to reaction zone 100 via the respective feeds 101, 102 to produce a crude ethanol product, e.g., stream 103. Crude ethanol product stream 103 is fed to a separation zone, e.g., distillation zone, 104, which yields ethanol product stream 105. Although FIG. 1 shows separation zone 104 in conjunction with reaction zone 100, which relates to acetic acid hydrogenation, separation zone 104 may be employed with other ethanol production processes. Depending on the reaction conditions, separation zone 104 may also yield water stream 106, unreacted acid stream 107, gas stream 108, and lights stream 109. In one embodiment, separation zone 104 comprises a plurality of separation columns.

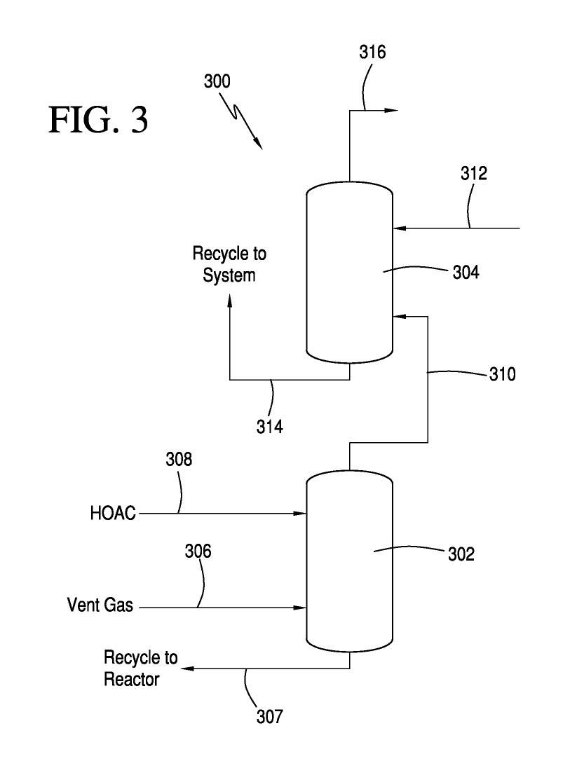

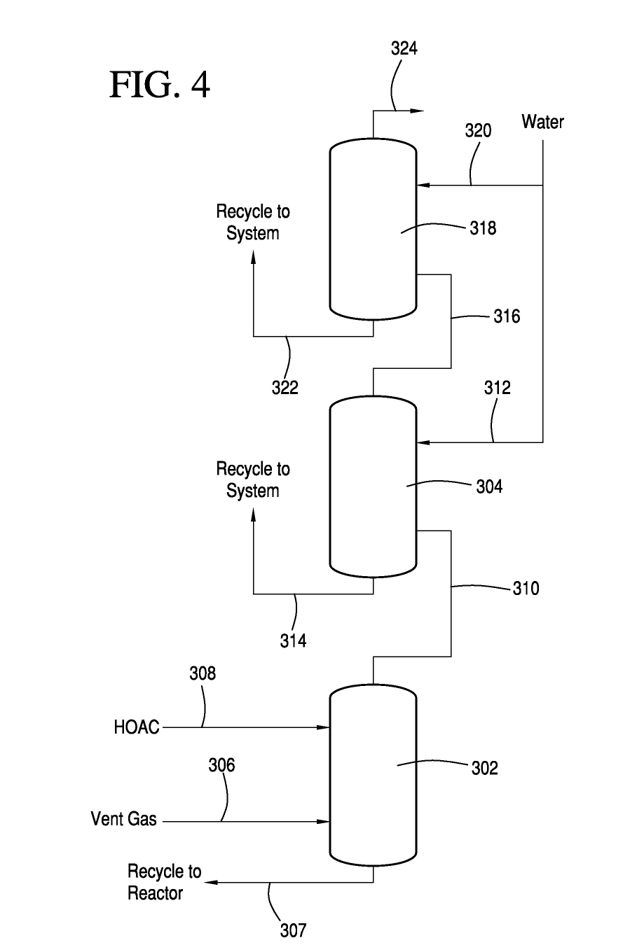

In one embodiment, one or more vent streams 110 may be withdrawn from the separation zone 104. Optionally, one or more vent streams 111 may be withdrawn from reaction zone 100. As shown in FIG. 1, multi-stage scrubbing unit may comprise one vessel having first stage 113 and second stage 114. In other embodiments, such as those shown in FIGS. 3 and 4, each stage may be configured in separate vessels.

The composition of vent stream(s) may vary depending on the reaction conditions and separation processes, but generally the vent streams comprise a major portion of non-condensable gases and a minor portion of volatile organics and/or entrained ethanol. For example, the non-condensable gases may be present in amounts greater than 1 wt. % based on the total weight of the vent streams, e.g., greater than 5 wt. % or greater than 10 wt. %. In terms of ranges, the non-condensable gases may be present in amounts from 0 to 99.9 wt. %, e.g. from 5 to 99.9 wt. % or from 10 to 99.9 wt. %. The volatile organics and/or entrained ethanol may be present in amounts less than 40 wt. % based on the total weight of the vent streams, e.g., less than 25 wt. % or less than 10 wt. %. In terms of ranges, the volatile organics and/or entrained ethanol may be present in amounts from 0 to 40 wt. %, e.g. from 0.01 to 25 wt. % or from 0.01 to 10 wt. %. Also, the vent streams may comprise water, which may be present in amounts less than 15 wt. % based on the total weight of the vent streams, e.g., less than 10 wt. % or less than 5 wt. %. In terms of ranges, the water may be present in amounts from 0 to 15 wt. %, e.g. from 0.1 to 10 wt. % or from 0.1 to 5 wt. %.

Vent stream(s) 110 and optional vent stream(s) 111 are fed to a lower portion of a multi-stage scrubbing unit 112, preferably fed to a lower portion of first stage 113 above exiting bottoms 116. First scrubbing solvent stream 115 is fed to an upper portion of the first stage 113. The temperature of the first scrubbing solvent ranges from 10° C. to 50° C., e.g., from 20° C. to 40° C. or from 25° C. to 40° C. Scrubbing solvent stream 115 may comprise an acidic solvent, and preferably comprises acetic acid. In one embodiment, a portion of the first scrubbing solvent may be obtained from the acetic acid fed to reaction zone 100 or from unreacted acid stream 107 from separation zone 104. Bottoms stream 116 is withdrawn from first stage 113, preferably continuously. Bottoms stream 116 may be enriched in acid as compared to vent streams 110 and/or 111. In one embodiment, bottoms stream 116 may be returned to reaction zone 100 to further produce ethanol from the recovered acetic acid.

Intermediate stream 117 is withdrawn from the upper portion of first stage 113 and fed to second stage 114. Second scrubbing solvent stream 118 is fed to the upper portion of second stage 114. The second scrubbing solvent is different from the first scrubbing solvent and preferably comprises an aqueous solvent, such as water. The temperature of the second scrubbing solvent ranges from 10° C. to 50° C., e.g., from 20° C. to 40° C. or from 25° C. to 40° C. In one embodiment, the water employed as the second scrubbing solvent may be obtained from water stream 106 that is separated in separation zone 104. Preferably, the water has a low impurity level of less than 5 wt. %, e.g. less than 3 wt. % or less than 1 wt. %, where impurities may refer to any organic compound in the water. The hydrogenation of acetic acid produces equal molar ratios of ethanol and water and thus there may be a sufficient amount of water from separation zone to scrub the vent streams 110 and/or 111. Intermediate stream 117 is scrubbed in second stage 114 to recover any remaining organics and/or entrained ethanol. Preferably the entire portion of intermediate stream 117 is fed to second stage 114, without purging any of intermediate stream 117. Return stream 119 may be withdrawn, preferably continuously, from the lower portion of second stage 114. In one embodiment, return stream 119 may be fed to separation zone 104 to further recover ethanol. Return stream 119 may also be returned in part to reaction zone 100. Scrubbed vent stream 120 is withdrawn from the top of multi-stage scrubbing unit 112 and removed from the hydrogenation system by a flare or by venting to the atmosphere.

As a result of the multiple scrubbing stages, the volatile organics and/or entrained ethanol may be effectively recovered from the vent stream(s). In one embodiment, the scrubbed vent stream(s) may comprise less than 5% of the total amount of volatile organics and/or entrained ethanol that were initially present in the vent stream(s), e.g., less than 3%, less than 1%, or less than 0.5%. For example, when the vent stream(s) comprise 10 wt. % volatile organics and/or entrained ethanol, the scrubbed vent stream may comprise less than 0.5 wt. % volatile organics and/or entrained ethanol. It is understood that the amount of volatile organics and/or entrained ethanol in vent stream(s) should be substantially recovered and the amounts of volatile organics and/or entrained ethanol in the scrubbed vent stream should generally be less than 2 wt. %, e.g. less than 1 wt. % or less than 0.5 wt. %. For the scrubbed vent stream, in terms of ranges, the total amounts of volatile organics and/or entrained ethanol, or each individual species, may be from 0.01 to 2 wt. %, e.g., from 0.01 to 1 wt. % or from 0.01 to 0.5 wt. %. In addition, the scrubbed vent stream comprises a majority of the non-condensable gases, e.g., up 99.9 wt. %.

In some embodiments, the volatile organics of the vent streams may comprise ethyl acetate, which is preferably recovered. Ethyl acetate may be recovered in bottoms stream 116 from the first stage 113 and/or in return stream 119 from second stage 114. The scrubbed vent stream preferably comprises substantially no ethyl acetate or comprises ethyl acetate in amounts of from 0.0001 to 0.5 wt. %, e.g., from 0.0001 to 0.3 wt. % or from 0.0001 to 0.1 wt. %.

The process of the present invention may be used with any ethanol production, preferably with ethanol produced by acetic acid hydrogenation. The materials, catalyst, reaction conditions, and separation are described further below.

The raw materials, acetic acid and hydrogen, used in connection with the process of this invention may be derived from any suitable source including natural gas, petroleum, coal, biomass, and so forth. As examples, acetic acid may be produced via methanol carbonylation, acetaldehyde oxidation, ethylene oxidation, oxidative fermentation, and anaerobic fermentation. As petroleum and natural gas prices fluctuate, becoming either more or less expensive, methods for producing acetic acid and intermediates such as methanol and carbon monoxide from alternate carbon sources have drawn increasing interest. In particular, when petroleum is relatively expensive compared to natural gas, it may become advantageous to produce acetic acid from synthesis gas (“syn gas”) that is derived from any available carbon source. U.S. Pat. No. 6,232,352, the disclosure of which is incorporated herein by reference, for example, teaches a method of retrofitting a methanol plant for the manufacture of acetic acid. By retrofitting a methanol plant, the large capital costs associated with CO generation for a new acetic acid plant are significantly reduced or largely eliminated. All or part of the syn gas is diverted from the methanol synthesis loop and supplied to a separator unit to recover CO and hydrogen, which are then used to produce acetic acid. In a similar manner, hydrogen for the hydrogenation step may be supplied from syn gas.

In some embodiments, some or all of the raw materials for the above-described acetic acid hydrogenation process may be derived partially or entirely from syngas. For example, the acetic acid may be formed from methanol and carbon monoxide, both of which may be derived from syngas. For example, the methanol may be formed by steam reforming syngas, and the carbon monoxide may be separated from syngas. Similarly, hydrogen that is used in the step of hydrogenating the acetic acid to form the crude ethanol product may be separated from syngas. The syngas, in turn, may be derived from variety of carbon sources. The carbon source, for example, may be selected from the group consisting of natural gas, oil, petroleum, coal, biomass, and combinations thereof.

Methanol carbonylation processes suitable for production of acetic acid are described in U.S. Pat. Nos. 7,208,624, 7,115,772, 7,005,541, 6,657,078, 6,627,770, 6,143,930, 5,599,976, 5,144,068, 5,026,908, 5,001,259, and 4,994,608, the entire disclosures of which are incorporated herein by reference. Optionally, the production of ethanol may be integrated with such methanol carbonylation processes.

U.S. Pat. No. RE 35,377 also incorporated herein by reference, provides a method for the production of methanol by conversion of carbonaceous materials such as oil, coal, natural gas and biomass materials. The process includes hydrogasification of solid and/or liquid carbonaceous materials to obtain a process gas which is steam pyrolized with additional natural gas to form synthesis gas. The syn gas is converted to methanol which may be carbonylated to acetic acid. The method likewise produces hydrogen which may be used in connection with this invention as noted above. U.S. Pat. No. 5,821,111, which discloses a process for converting waste biomass through gasification into synthesis gas, and U.S. Pat. No. 6,685,754, which discloses a method for the production of a hydrogen-containing gas composition, such as a synthesis gas including hydrogen and carbon monoxide, are incorporated herein by reference in their entireties.

The acetic acid fed to the hydrogenation reaction may also comprise other carboxylic acids and anhydrides, as well as acetaldehyde and acetone. Preferably, a suitable acetic acid feed stream comprises one or more of the compounds selected from the group consisting of acetic acid, acetic anhydride, acetaldehyde, ethyl acetate, and mixtures thereof. These other compounds may also be hydrogenated in the processes of the present invention. In some embodiments, the presence of carboxylic acids, such as propanoic acid or its anhydride, may be beneficial in producing propanol. Water may also be present in the acetic acid feed.

Alternatively, acetic acid in vapor form may be taken directly as crude product from the flash vessel of a methanol carbonylation unit of the class described in U.S. Pat. No. 6,657,078, the entirety of which is incorporated herein by reference. The crude vapor product, for example, may be fed directly to the ethanol synthesis reaction zones of the present invention without the need for condensing the acetic acid and light ends or removing water, saving overall processing costs.

The acetic acid may be vaporized at the reaction temperature, following which the vaporized acetic acid can be fed along with hydrogen in an undiluted state or diluted with a relatively inert carrier gas, such as nitrogen, argon, helium, carbon dioxide and the like. For reactions run in the vapor phase, the temperature should be controlled in the system such that it does not fall below the dew point of acetic acid. In one embodiment the acetic acid may be vaporized at the boiling point of acetic acid at the particular pressure, and then the vaporized acetic acid may be further heated to the reactor inlet temperature. In another embodiment, the acetic acid is transferred to the vapor state by passing hydrogen, recycle gas, another suitable gas, or mixtures thereof through the acetic acid at a temperature below the boiling point of acetic acid, thereby humidifying the carrier gas with acetic acid vapors, followed by heating the mixed vapors up to the reactor inlet temperature. Preferably, the acetic acid is transferred to the vapor state by passing hydrogen and/or recycle gas through the acetic acid at a temperature at or below 125° C., followed by heating of the combined gaseous stream to the reactor inlet temperature.

Some embodiments of the process of hydrogenating acetic acid to form ethanol according to one embodiment of the invention may include a variety of configurations using a fixed bed reactor or a fluidized bed reactor. In many embodiments of the present invention, an “adiabatic” reactor can be used; that is, there is little or no need for internal plumbing through the reaction zone to add or remove heat. In other embodiments, radial flow reactor or reactors may be employed, or a series of reactors may be employed with or with out heat exchange, quenching, or introduction of additional feed material. Alternatively, a shell and tube reactor provided with a heat transfer medium may be used. In many cases, the reaction zone may be housed in a single vessel or in a series of vessels with heat exchangers therebetween.

In preferred embodiments, the catalyst is employed in a fixed bed reactor, e.g., in the shape of a pipe or tube, where the reactants, typically in the vapor form, are passed over or through the catalyst. Other reactors, such as fluid or ebullient bed reactors, can be employed. In some instances, the hydrogenation catalysts may be used in conjunction with an inert material to regulate the pressure drop of the reactant stream through the catalyst bed and the contact time of the reactant compounds with the catalyst particles.

The hydrogenation reaction may be carried out in either the liquid phase or vapor phase. Preferably, the reaction is carried out in the vapor phase under the following conditions. The reaction temperature may range from 125° C. to 350° C., e.g., from 200° C. to 325° C., from 225° C. to 300° C., or from 250° C. to 300° C. The pressure may range from 10 KPa to 3000 KPa (about 1.5 to 435 psi), e.g., from 50 KPa to 2300 KPa, or from 100 KPa to 1500 KPa. The reactants may be fed to the reactor at a gas hourly space velocity (GHSV) of greater than 500 hr⁻¹, e.g., greater than 1000 hr⁻¹, greater than 2500 hr⁻¹ or even greater than 5000 hr⁻¹. In terms of ranges the GHSV may range from 50 hr⁻¹ to 50,000 hr⁻¹, e.g., from 500 hr₋₁ to 30,000 hr⁻¹, from 1000 hr⁻¹ to 10,000 hr⁻¹, or from 1000 hr⁻¹ to 6500 hr⁻¹.

The hydrogenation optionally is carried out at a pressure just sufficient to overcome the pressure drop across the catalytic bed at the GHSV selected, although there is no bar to the use of higher pressures, it being understood that considerable pressure drop through the reactor bed may be experienced at high space velocities, e.g., 5000 hr⁻¹ or 6,500 hr⁻¹.

Although the reaction consumes two moles of hydrogen per mole of acetic acid to produce one mole of ethanol, the actual molar ratio of hydrogen to acetic acid in the feed stream may vary from about 100:1 to 1:100, e.g., from 50:1 to 1:50, from 20:1 to 1:2, or from 12:1 to 1:1. Most preferably, the molar ratio of hydrogen to acetic acid is greater than 2:1, e.g., greater than 4:1 or greater than 8:1.

Contact or residence time can also vary widely, depending upon such variables as amount of acetic acid, catalyst, reactor, temperature and pressure. Typical contact times range from a fraction of a second to more than several hours when a catalyst system other than a fixed bed is used, with preferred contact times, at least for vapor phase reactions, of from 0.1 to 100 seconds, e.g., from 0.3 to 80 seconds or from 0.4 to 30 seconds.

The hydrogenation of acetic acid to form ethanol is preferably conducted in the presence of a hydrogenation catalyst. Suitable hydrogenation catalysts include catalysts comprising a first metal and optionally one or more of a second metal, a third metal or any number of additional metals, optionally on a catalyst support. The first and optional second and third metals may be selected from Group IB, IIB, IIIB, IVB, VB, VIIB, VIIB, VIII transition metals, a lanthanide metal, an actinide metal, or a metal selected from any of Groups IIIA, IVA, VA, and VIA. Preferred metal combinations for some exemplary catalyst compositions include platinum/tin, platinum/ruthenium, platinum/rhenium, palladium/ruthenium, palladium/rhenium, cobalt/palladium, cobalt/platinum, cobalt/chromium, cobalt/ruthenium, silver/palladium, copper/palladium, nickel/palladium, gold/palladium, ruthenium/rhenium, and ruthenium/iron. Exemplary catalysts are further described in U.S. Pat. Nos. 7,608,744 and 7,863,489 and U.S. Pub. No. 2010/0197485, the entireties of which are incorporated herein by reference.

In one embodiment, the catalyst comprises a first metal selected from the group consisting of copper, iron, cobalt, nickel, ruthenium, rhodium, palladium, osmium, iridium, platinum, titanium, zinc, chromium, rhenium, molybdenum, and tungsten. Preferably, the first metal is selected from the group consisting of platinum, palladium, cobalt, nickel, and ruthenium. More preferably, the first metal is selected from platinum and palladium. In embodiments of the invention where the first metal comprises platinum, it is preferred that the catalyst comprises platinum in an amount less than 5 wt. %, e.g., less than 3 wt. % or less than 1 wt. %, due to the high commercial demand for platinum.

As indicated above, in some embodiments, the catalyst further comprises a second metal, which typically would function as a promoter. If present, the second metal preferably is selected from the group consisting of copper, molybdenum, tin, chromium, iron, cobalt, vanadium, tungsten, palladium, platinum, lanthanum, cerium, manganese, ruthenium, rhenium, gold, and nickel. More preferably, the second metal is selected from the group consisting of copper, tin, cobalt, rhenium, and nickel. Most preferably, the second metal is selected from tin and rhenium.

In certain embodiments where the catalyst includes two or more metals, e.g., a first metal and a second metal, the first metal is present in the catalyst in an amount from 0.1 to 10 wt. %, e.g., from 0.1 to 5 wt. %, or from 0.1 to 3 wt. %. The second metal preferably is present in an amount from 0.1 and 20 wt. %, e.g., from 0.1 to 10 wt. %, or from 0.1 to 5 wt. %. For catalysts comprising two or more metals, the two or more metals may be alloyed with one another, or may comprise a non-alloyed metal solution or mixture.

The preferred metal ratios may vary depending on the metals used in the catalyst. In some exemplary embodiments, the mole ratio of the first metal to the second metal is from 10:1 to 1:10, e.g., from 4:1 to 1:4, from 2:1 to 1:2, from 1.5:1 to 1:1.5 or from 1.1:1 to 1:1.1.

The catalyst may also comprise a third metal selected from any of the metals listed above in connection with the first or second metal, so long as the third metal is different from both the first and second metals. In preferred embodiments, the third metal is selected from the group consisting of cobalt, palladium, ruthenium, copper, zinc, platinum, tin, and rhenium. More preferably, the third metal is selected from cobalt, palladium, and ruthenium. When present, the total weight of the third metal is preferably from 0.05 to 4 wt. %, e.g., from 0.1 to 3 wt. %, or from 0.1 to 2 wt. %.

In addition to one or more metals, in some embodiments of the present invention, the catalysts further comprise a support or a modified support. As used herein, the term “modified support” refers to a support that includes a support material and a support modifier, which adjusts the acidity of the support material.

The total weight of the support or modified support, based on the total weight of the catalyst, preferably is from 75 to 99.9 wt. %, e.g., from 78 to 97 wt. %, or from 80 to 95 wt. %. In preferred embodiments that utilize a modified support, the support modifier is present in an amount from 0.1 to 50 wt. %, e.g., from 0.2 to 25 wt. %, from 0.5 to 15 wt. %, or from 1 to 8 wt. %, based on the total weight of the catalyst. The metals of the catalysts may be dispersed throughout the support, layered throughout the support, coated on the outer surface of the support (i.e., egg shell), or decorated on the surface of the support.

As will be appreciated by those of ordinary skill in the art, support materials are selected such that the catalyst system is suitably active, selective and robust under the process conditions employed for the formation of ethanol.

Suitable support materials may include, for example, stable metal oxide-based supports or ceramic-based supports. Preferred supports include silicaceous supports, such as silica, silica/alumina, a Group IIA silicate such as calcium metasilicate, pyrogenic silica, high purity silica, and mixtures thereof. Other supports may include, but are not limited to, iron oxide, alumina, titania, zirconia, magnesium oxide, carbon, graphite, high surface area graphitized carbon, activated carbons, and mixtures thereof.

As indicated, the catalyst support may be modified with a support modifier. In some embodiments, the support modifier may be an acidic modifier that increases the acidity of the catalyst. Suitable acidic support modifiers may be selected from the group consisting of: oxides of Group IVB metals, oxides of Group VB metals, oxides of Group VIB metals, oxides of Group VIIB metals, oxides of Group VIIIB metals, aluminum oxides, and mixtures thereof. Acidic support modifiers include those selected from the group consisting of TiO₂, ZrO₂, Nb₂O₅, Ta₂O₅, Al₂O₃, B₂O₃, P₂O₅, and Sb₂O₃. Preferred acidic support modifiers include those selected from the group consisting of TiO₂, ZrO₂, Nb₂O₅, Ta₂O₅, and Al₂O₃. The acidic modifier may also include WO₃, MoO₃, Fe₂O₃, Cr₂O₃, V₂O₅, MnO₂, CuO, Co₂O₃, Bi₂O₃.

In another embodiments, the support modifier may be a basic modifier that has a low volatility or no volatility. Such basic modifiers, for example, may be selected from the group consisting of: (i) alkaline earth oxides, (ii) alkali metal oxides, (iii) alkaline earth metal metasilicates, (iv) alkali metal metasilicates, (v) Group IIB metal oxides, (vi) Group IIB metal metasilicates, (vii) Group IIIB metal oxides, (viii) Group IIIB metal metasilicates, and mixtures thereof. In addition to oxides and metasilicates, other types of modifiers including nitrates, nitrites, acetates, and lactates may be used. The basic support modifier may be selected from the group consisting of oxides and metasilicates of any of sodium, potassium, magnesium, calcium, scandium, yttrium, and zinc, as well as mixtures of any of the foregoing. More preferably, the basic support modifier is a calcium silicate, and even more preferably calcium metasilicate (CaSiO₃). If the basic support modifier comprises calcium metasilicate, it is preferred that at least a portion of the calcium metasilicate is in crystalline form.

A preferred silica support material is SS61138 High Surface Area (HSA) Silica Catalyst Carrier from Saint Gobain NorPro. The Saint-Gobain NorPro SS61138 silica exhibits the following properties: contains approximately 95 wt. % high surface area silica; surface area of about 250 m²/g; median pore diameter of about 12 nm; average pore volume of about 1.0 cm³/g as measured by mercury intrusion porosimetry; and packing density of about 0.352 g/cm³ (22 lb/ft³).

A preferred silica/alumina support material is KA-160 silica spheres from Süd-Chemie having a nominal diameter of about 5 mm, a density of about 0.562 g/ml, an absorptivity of about 0.583 g H₂O/g support, a surface area of about 160 to 175 m²/g, and a pore volume of about 0.68 ml/g.

The catalyst compositions suitable for use with the present invention preferably are formed through metal impregnation of the modified support, although other processes such as chemical vapor deposition may also be employed. Such impregnation techniques are described in U.S. Pat. Nos. 7,608,744 and 7,863,489 and U.S. Pub. No. 2010/0197485 referred to above, the entireties of which are incorporated herein by reference.

In particular, the hydrogenation of acetic acid may achieve favorable conversion of acetic acid and favorable selectivity and productivity to ethanol. For purposes of the present invention, the term “conversion” refers to the amount of acetic acid in the feed that is converted to a compound other than acetic acid. Conversion is expressed as a mole percentage based on acetic acid in the feed. The conversion may be at least 10%, e.g., at least 20%, at least 40%, at least 50%, at least 60%, at least 70% or at least 80%. Although catalysts that have high conversions are desirable, such as at least 80% or at least 90%, in some embodiments, a low conversion may be acceptable at high selectivity for ethanol. It is, of course, well understood that in many cases, it is possible to compensate for conversion by appropriate recycle streams or use of larger reactors, but it is more difficult to compensate for poor selectivity.

Selectivity is expressed as a mole percent based on converted acetic acid. It should be understood that each compound converted from acetic acid has an independent selectivity and that selectivity is independent from conversion. For example, if 60 mole % of the converted acetic acid is converted to ethanol, we refer to the ethanol selectivity as 60%. Preferably, the catalyst selectivity to ethoxylates is at least 60%, e.g., at least 70%, or at least 80%. As used herein, the term “ethoxylates” refers specifically to the compounds ethanol, acetaldehyde, and ethyl acetate. Preferably, the selectivity to ethanol is at least 80%, e.g., at least 85% or at least 88%. Preferred embodiments of the hydrogenation process also have low selectivity to undesirable products, such as methane, ethane, and carbon dioxide. The selectivity to these undesirable products preferably is less than 4%, e.g., less than 2% or less than 1%. More preferably, these undesirable products are present in undetectable amounts. Formation of alkanes may be low, and ideally less than 2%, less than 1%, or less than 0.5% of the acetic acid passed over the catalyst is converted to alkanes, which have little value other than as fuel.

The term “productivity,” as used herein, refers to the grams of a specified product, e.g., ethanol, formed during the hydrogenation based on the kilograms of catalyst used per hour. A productivity of at least 200 grams of ethanol per kilogram catalyst per hour, e.g., at least 400 grams of ethanol per kilogram catalyst per hour, or at least 600 grams of ethanol per kilogram catalyst per hour, is preferred. In terms of ranges, the productivity preferably is from 200 to 3,000 grams of ethanol per kilogram catalyst per hour, e.g., from 400 to 2,500 per kilogram catalyst per hour or from 600 to 2,000 per kilogram catalyst per hour.

Operating under the conditions of the present invention may result in ethanol production on the order of at least 0.1 tons of ethanol per hour, e.g., at least 1 ton of ethanol per hour, at least 5 tons of ethanol per hour, or at least 10 tons of ethanol per hour. Larger scale industrial production of ethanol, depending on the scale, generally should be at least 1 ton of ethanol per hour, e.g., at least 15 tons of ethanol per hour or at least 30 tons of ethanol per hour. In terms of ranges, for large scale industrial production of ethanol, the process of the present invention may produce from 0.1 to 160 tons of ethanol per hour, e.g., from 15 to 160 tons of ethanol per hour or from 30 to 80 tons of ethanol per hour. Ethanol production from fermentation, due the economies of scale, typically does not permit the single facility ethanol production that may be achievable by employing embodiments of the present invention.

In various embodiments of the present invention, the crude ethanol product produced by the hydrogenation process, before any subsequent processing, such as purification and separation, will typically comprise unreacted acetic acid, ethanol and water. As used herein, the term “crude ethanol product” refers to any composition comprising from 5 to 70 wt. % ethanol and from 5 to 35 wt. % water. In some exemplary embodiments, the crude ethanol product comprises ethanol in an amount from 5 wt. % to 70 wt. %, e.g., from 10 wt. % to 60 wt. %, or from 15 wt. % to 50 wt. %, based on the total weight of the crude ethanol product. Preferably, the crude ethanol product contains at least 10 wt. % ethanol, at least 15 wt. % ethanol or at least 20 wt. % ethanol. The crude ethanol product typically will further comprise unreacted acetic acid, depending on conversion, for example, in an amount of less than 90 wt. %, e.g., less than 80 wt. % or less than 70 wt. %. In terms of ranges, the unreacted acetic acid optionally is present in the crude ethanol product in an amount from 0 to 90 wt. %, e.g., from 5 to 80 wt. %, from 15 to 70 wt. %, from 20 to 70 wt. % or from 25 to 65 wt. %. As water is formed in the reaction process, water will generally be present in the crude ethanol product, for example, in amounts ranging from 5 to 35 wt. %, e.g., from 10 to 30 wt. % or from 10 to 26 wt. %.

Ethyl acetate may also be produced during the hydrogenation of acetic acid, or through side reactions and may be present, for example, in amounts ranging from 0 to 20 wt. %, e.g., from 0 to 15 wt. %, from 1 to 12 wt. % or from 3 to 10 wt. %. In addition, acetaldehyde may be produced through side reactions, and may be present, for example, in amounts ranging from 0 to 10 wt. %, e.g., from 0 to 3 wt. %, from 0.1 to 3 wt. % or from 0.2 to 2 wt. %. Other components, such as, for example, alcohols, esters, ethers, aldehydes, ketones, alkanes, and carbon dioxide, if detectable, collectively may be present in amounts less than 10 wt. %, e.g., less than 6 wt. % or less than 4 wt. %. In terms of ranges, these other components may be present in an amount from 0.1 to 10 wt. %, e.g., from 0.1 to 6 wt. %, or from 0.1 to 4 wt. %. Exemplary component ranges for the crude ethanol product are provided in Table 1.

TABLE 1
CRUDE ETHANOL PRODUCT COMPOSITIONS

| | Conc. | Conc. | Conc. | Conc. |
|---|---|---|---|---|
| Component | (wt. %) | (wt. %) | (wt. %) | (wt. %) |
| Ethanol | 5 to 70 | 10 to 60 | 15 to 50 | 25 to 50 |
| Acetic Acid | 0 to 90 | 5 to 80 | 15 to 70 | 20 to 70 |
| Water | 5 to 35 | 5 to 30 | 10 to 30 | 10 to 26 |
| Ethyl Acetate | 0 to 20 | 0 to 15 | 1 to 12 | 3 to 10 |
| Acetaldehyde | 0 to 10 | 0 to 3 | 0.1 to 3 | 0.2 to 2 |
| Others | 0.1 to 10 | 0.1 to 6 | 0.1 to 4 | — |

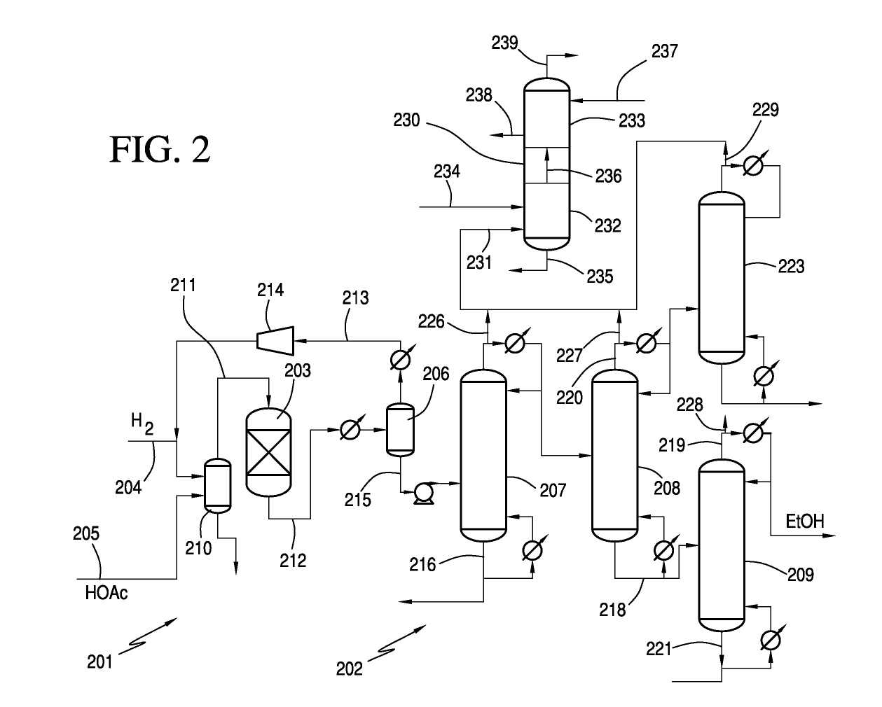

FIG. 2 shows an exemplary hydrogenation system 200 suitable for the hydrogenation of acetic acid and separating ethanol from the crude reaction mixture according to one embodiment of the invention. System 200 comprises reaction zone 201 and separation zone 202. Reaction zone 201 comprises reactor 203, hydrogen feed line 204 and acetic acid feed line 205. Separation zone 202 comprises flasher 206, first column 207, second column 208, third column 209, and fourth column 223. Hydrogen and acetic acid are fed to a vaporizer 210 via lines 204 and 205, respectively, to create a vapor feed stream in line 211 that is directed to reactor 203. In one embodiment, lines 204 and 205 may be combined and jointly fed to the vaporizer 210. The temperature of the vapor feed stream in line 211 is preferably from 100° C. to 350° C., e.g., from 120° C. to 310° C. or from 150° C. to 300° C. Any feed that is not vaporized is removed from vaporizer 210, as shown in FIG. 2, and may be recycled thereto. In addition, although FIG. 2 shows line 211 being directed to the top of reactor 203, line 211 may be directed to the side, upper portion, or bottom of reactor 203. Further modifications and additional components to reaction zone 201 are described herein.

Reactor 203 contains the catalyst that is used in the hydrogenation of the carboxylic acid, preferably acetic acid. In one embodiment, one or more guard beds (not shown) may be used to protect the catalyst from poisons or undesirable impurities contained in the feed or return/recycle streams. Such guard beds may be employed in the vapor or liquid streams. Suitable guard bed materials are known in the art and include, for example, carbon, silica, alumina, ceramic, or resins. In one aspect, the guard bed media is functionalized to trap particular species such as sulfur or halogens. During the hydrogenation process, a crude ethanol product stream is withdrawn, preferably continuously, from reactor 203 via line 212.

The crude ethanol product stream may be condensed and fed to flasher 206, which, in turn, provides a vapor stream and a liquid stream. The flasher 206 in one embodiment preferably operates at a temperature of from 50° C. to 500° C., e.g., from 70° C. to 400° C. or from 100° C. to 350° C. In one embodiment, the pressure of flasher 206 preferably is from 50 KPa to 2000 KPa, e.g., from 75 KPa to 1500 KPa or from 100 to 1000 KPa. In one preferred embodiment the temperature and pressure of the flasher is similar to the temperature and pressure of the reactor 203.

The vapor stream exiting the flasher 206 may comprise hydrogen and hydrocarbons, which may be purged and/or returned to reaction zone 201 via line 213. As shown in FIG. 2, the returned portion of the vapor stream passes through compressor 214 and is combined with the hydrogen feed and co-fed to vaporizer 210.

The liquid from flasher 206 is withdrawn and pumped as a feed composition via line 215 to the side of first column 207, also referred to as an acid separation column. The contents of line 215 typically will be substantially similar to the product obtained directly from the reactor, and may, in fact, also be characterized as a crude ethanol product. However, the feed composition in line 215 preferably has less hydrogen, carbon dioxide, methane or ethane, which are removed by flasher 206. Exemplary components of the liquid in line 215 are provided in Table 2. It should be understood that liquid line 215 may contain other components, not listed, such as components in the feed.

TABLE 2
FEED COMPOSITION

| | Conc. | Conc. | Conc. |
|---|---|---|---|
| | (wt. %) | (wt. %) | (wt. %) |
| Ethanol | 5 to 70 | 10 to 60 | 15 to 50 |
| Acetic Acid | <90 | 5 to 80 | 15 to 70 |
| Water | 5 to 35 | 5 to 30 | 10 to 30 |
| Ethyl Acetate | <20 | 0.001 to 15 | 1 to 12 |
| Acetaldehyde | <10 | 0.001 to 3 | 0.1 to 3 |
| Acetal | <5 | 0.001 to 2 | 0.005 to 1 |
| Acetone | <5 | 0.0005 to 0.05 | 0.001 to 0.03 |
| Other Esters | <5 | <0.005 | <0.001 |
| Other Ethers | <5 | <0.005 | <0.001 |
| Other Alcohols | <5 | <0.005 | <0.001 |

The amounts indicated as less than (<) in the tables throughout present application are preferably not present and if present may be present in trace amounts or in amounts greater than 0.0001 wt. %.

The “other esters” in Table 2 may include, but are not limited to, ethyl propionate, methyl acetate, isopropyl acetate, n-propyl acetate, n-butyl acetate or mixtures thereof. The “other ethers” in Table 2 may include, but are not limited to, diethyl ether, methyl ethyl ether, isobutyl ethyl ether or mixtures thereof. The “other alcohols” in Table 2 may include, but are not limited to, methanol, isopropanol, n-propanol, n-butanol or mixtures thereof. In one embodiment, the feed composition, e.g., line 215, may comprise propanol, e.g., isopropanol and/or n-propanol, in an amount from 0.001 to 0.1 wt. %, from 0.001 to 0.05 wt. % or from 0.001 to 0.03 wt. %. It should be understood that these other components may be carried through in any of the distillate or residue streams described herein and will not be further described herein, unless indicated otherwise.

Optionally, the crude ethanol product may pass through one or more membranes to separate hydrogen and/or other non-condensable gases. In other optional embodiments, the crude ethanol product may be fed directly to the acid separation column as a vapor feed and the non-condensable gases may be recovered from the overhead of the column.

When the content of acetic acid in line 215 is less than 5 wt. %, the acid separation column 207 may be skipped and line 215 may be introduced directly to second column 208, also referred to herein as a “light ends column.”

In the embodiment shown in FIG. 2, line 215 is introduced in the lower part of first column 207, e.g., lower half or lower third. Depending on the acetic acid conversion and operation of column 207, unreacted acetic acid, water, and other heavy components, if present, are removed from the composition in line 215 and are withdrawn, preferably continuously, as residue. In some embodiments, especially with higher conversions of acetic acid of at least 80%, or at least 90%, it may be beneficially to remove a majority of water in line 215 along with substantially all the acetic acid in residue stream 216. Residue stream 216 may be recycled to reaction zone 201. In addition, a portion of the water in residue stream 216 may be separated and purged with the acid rich portion being returned to reaction zone 201. In other embodiments, the residue stream 216 may be a dilute acid stream that may be treated in a weak acid recovery system or sent to a reactive distillation column to convert the acid to esters.

In one embodiment, a portion of the first residue stream 216 may be fed to multi-stage scrubbing unit 230 as the acidic solvent or aqueous solvent. Depending on the concentration of acid in the first residue stream 216, the acid and water may be separated and fed a separate scrubbing solvents to the multi-stage scrubbing unit. When first residue stream 216 comprises an acid rich stream comprising greater than 60 wt. % acetic acid, first residue stream 216 may be the acidic solvent. When first residue stream 216 comprises a dilute acid stream, e.g., comprising less than 60 wt. % acetic acid, first residue stream 216 may be the aqueous solvent.

First column 207 also forms an overhead distillate, which is withdrawn in line 217, and which may be condensed and refluxed, for example, at a ratio of from 10:1 to 1:10, e.g., from 3:1 to 1:3 or from 1:2 to 2:1.

The columns shown in the FIGS. may comprise any distillation column capable of performing the desired separation and/or purification. Each column preferably comprises a tray column having from 1 to 150 trays, e.g., from 10 to 100, from 20 to 95 trays or from 30 to 75 trays. The trays may be sieve trays, fixed valve trays, movable valve trays, or any other suitable design known in the art. In other embodiments, a packed column may be used. For packed columns, structured packing or random packing may be employed. The trays or packing may be arranged in one continuous column, or they may be arranged in two or more columns such that the vapor from the first section enters the second section while the liquid from the second section enters the first section.

The associated condensers and liquid separation vessels that may be employed with each of the distillation columns may be of any conventional design and are simplified in FIG. 2. As shown in FIG. 2, heat may be supplied to the base of each column or to a circulating bottom stream through a heat exchanger or reboiler. Other types of reboilers, such as internal reboilers, may also be used. The heat that is provided to the reboilers may be derived from any heat generated during the process that is integrated with the reboilers or from an external source such as another heat generating chemical process or a boiler. Although one reactor and one flasher are shown in FIG. 2, additional reactors, flashers, condensers, heating elements, and other components may be used in various embodiments of the present invention. As will be recognized by those skilled in the art, various condensers, pumps, compressors, reboilers, drums, valves, connectors, separation vessels, etc., normally employed in carrying out chemical processes may also be combined and employed in the processes of the present invention.

The temperatures and pressures employed in the columns 207, 208, 209, or 223 may vary. As a practical matter, pressures from 10 KPa to 3000 KPa will generally be employed in these zones although, in some embodiments, subatmospheric pressures or superatomic pressures may be employed. Temperatures within the various zones will normally range between the boiling points of the composition removed as the distillate and the composition removed as the residue. As will be recognized by those skilled in the art, the temperature at a given location in an operating distillation column is dependent on the composition of the material at that location and the pressure of column. In addition, feed rates may vary depending on the size of the production process and, if described, may be generically referred to in terms of feed weight ratios.

When column 207 is operated under standard atmospheric pressure, the temperature of the residue exiting in line 216 from column 207 preferably is from 95° C. to 120° C., e.g., from 105° C. to 117° C. or from 110° C. to 115° C. The temperature of the distillate exiting in line 217 from column 207 preferably is from 70° C. to 110° C., e.g., from 75° C. to 95° C. or from 80° C. to 90° C. In other embodiments, the pressure of first column 207 may range from 0.1 KPa to 510 KPa, e.g., from 1 KPa to 475 KPa or from 1 KPa to 375 KPa. In one exemplary embodiment a distillate and residue compositions for first column 207 are provided in Table 3 below. Note that these compositions may change depending on acetic acid conversion, the operation of the column and whether a majority of the water is removed in the residue. For convenience, the distillate and residue of the first column may also be referred to as the “first distillate” or “first residue.” The distillates or residues of the other columns may also be referred to with similar numeric modifiers (second, third, etc.) in order to distinguish them from one another, but such modifiers should not be construed as requiring any particular separation order.

TABLE 3
FIRST COLUMN

| | Conc. | Conc. | Conc. |
|---|---|---|---|
| | (wt. %) | (wt. %) | (wt. %) |
| Distillate | | | |
| Ethanol | 20 to 75 | 30 to 70 | 40 to 65 |
| Water | 10 to 40 | 15 to 35 | 20 to 35 |
| Acetic Acid | <2 | 0.001 to 0.5 | 0.01 to 0.2 |
| Ethyl Acetate | <60 | 5.0 to 40 | 10 to 30 |
| Acetaldehyde | <10 | 0.001 to 5 | 0.01 to 4 |
| Acetal | <0.1 | <0.1 | <0.05 |
| Acetone | <0.05 | 0.001 to 0.03 | 0.01 to 0.025 |
| Residue | | | |
| Acetic Acid | 60 to 100 | 70 to 95 | 85 to 92 |
| Water | <30 | 1 to 20 | 1 to 15 |
| Ethanol | <1 | <0.9 | <0.07 |

Some species, such as acetals, may decompose in column 207 to low or even no detectable amounts. In addition, there may be a non-catalyzed equilibrium reaction after the crude ethanol product 212 exits the reactor 203 in liquid feed 215. Depending on the concentration of acetic acid, the equilibrium may be driven towards formation of ethyl acetate. The equilibrium may be regulated using the residence time and/or temperature of liquid feed 215.

In some embodiments, the crude ethanol product is temporarily stored, e.g., in a holding tank (not shown), prior to being directed to separation zone 102. In addition, holding tanks (not shown) may be included between the reaction zone 201 and separation zone 202 and/or between the various units of reaction zone 201 and/or the separation zone 202 for temporarily storing components. In these cases, the holding tanks may be vented to regulate pressure. The vent streams that result from this venting may comprise volatile organics and/or entrained ethanol, which, in preferred embodiments, may be directed to a multi-stage scrubbing unit 230 for further recovery.

The distillate, e.g., overhead stream, of column 207 optionally is condensed and refluxed as shown in FIG. 2, preferably, at a reflux ratio of 1:5 to 10:1. The distillate in line 217 preferably comprises ethanol, ethyl acetate, and water, along with other impurities, which may be difficult to separate due to the formation of binary and tertiary azeotropes.

The first distillate in line 217 is introduced to the second column 208, also referred to as the “light ends column,” preferably in the middle part of column 208, e.g., middle half or middle third. Second column 208 may be a tray column or packed column. In one embodiment, second column 208 is a tray column having from 5 to 70 trays, e.g., from 15 to 50 trays, or from 20 to 45 trays. As one example, when a 25 tray column is used in a column without water extraction, line 217 is introduced at tray 17. In one embodiment, the second column 208 may be an extractive distillation column. In such embodiments, an extraction agent, such as water, may be added to second column 208. If the extraction agent comprises water, it may be obtained from an external source or from an internal return/recycle line from one or more of the other columns.

Although the temperature and pressure of second column 208 may vary, when at atmospheric pressure, the temperature of the second residue exiting in line 218 from second column 208 preferably is from 60° C. to 90° C., e.g., from 70° C. to 90° C. or from 80° C. to 90° C. The temperature of the second distillate exiting in line 220 from second column 208 preferably is from 50° C. to 90° C., e.g., from 60° C. to 80° C. or from 60° C. to 70° C. Column 208 may operate at atmospheric pressure. In other embodiments, the pressure of second column 208 may range from 0.1 KPa to 510 KPa, e.g., from 1 KPa to 475 KPa or from 1 KPa to 375 KPa. Exemplary distillate and residue compositions for second column 208 are provided in Table 4 below. It should be understood that the distillate and residue may also contain other components, not listed, such as components in the feed.

TABLE 4
SECOND COLUMN

| | Conc. | Conc. | Conc. |
|---|---|---|---|
| | (wt. %) | (wt. %) | (wt. %) |
| Distillate | | | |
| Ethyl Acetate | 10 to 90 | 25 to 90 | 50 to 90 |
| Acetaldehyde | 1 to 25 | 1 to 15 | 1 to 8 |
| Water | 1 to 25 | 1 to 20 | 4 to 16 |
| Ethanol | <30 | 0.001 to 15 | 0.01 to 5 |
| Acetal | <5 | 0.001 to 2 | 0.01 to 1 |
| Residue | | | |
| Water | 30 to 70 | 30 to 60 | 30 to 50 |
| Ethanol | 20 to 75 | 30 to 70 | 40 to 70 |
| Ethyl Acetate | <3 | 0.001 to 2 | 0.001 to 0.5 |
| Acetic Acid | <0.5 | 0.001 to 0.3 | 0.001 to 0.2 |

The weight ratio of ethanol in the second residue to ethanol in the second distillate preferably is at least 3:1, e.g., at least 6:1, at least 8:1, at least 10:1 or at least 15:1. The weight ratio of ethyl acetate in the second residue to ethyl acetate in the second distillate preferably is less than 0.4:1, e.g., less than 0.2:1 or less than 0.1:1. In embodiments that use an extractive column with water as an extraction agent as the second column 208, the weight ratio of ethyl acetate in the second residue to ethyl acetate in the second distillate approaches zero.

As shown in FIG. 2, the second residue from the bottom of second column 208, which comprises ethanol and water, is fed via line 218 to third column 209, also referred to as a “product column.” More preferably, the second residue in line 218 is introduced in the lower part of third column 209, e.g., lower half or lower third. Third column 209 recovers ethanol, which preferably is substantially pure other than the azeotropic water content, as the distillate in line 219. The distillate of third column 209 preferably is refluxed as shown in FIG. 2, for example, at a reflux ratio of from 1:10 to 10:1, e.g., from 1:3 to 3:1 or from 1:2 to 2:1. The third residue in line 221, which preferably comprises primarily water, preferably is removed from the system 200 or may be partially returned to any portion of the system 200, e.g., a multi-stage scrubber 230. The third residue in line 221 may also be used as an extractive agent in the second column 208 or to hydrolyze a stream comprising ethyl acetate.

Third column 209 is preferably a tray column as described above and preferably operates at atmospheric pressure. The temperature of the third distillate exiting in line 219 from third column 209 preferably is from 60° C. to 110° C., e.g., from 70° C. to 100° C. or from 75° C. to 95° C. The temperature of the third residue exiting from third column 209 preferably is from 70° C. to 115° C., e.g., from 80° C. to 110° C. or from 85° C. to 105° C., when the column is operated at atmospheric pressure. Exemplary components of the distillate and residue compositions for third column 109 are provided in Table 5 below. It should be understood that the distillate and residue may also contain other components, not listed, such as components in the feed.

TABLE 5
THIRD COLUMN

| | Conc. | Conc. | Conc. |
|---|---|---|---|
| | (wt. %) | (wt. %) | (wt. %) |
| Distillate | | | |
| Ethanol | 75 to 96 | 80 to 96 | 85 to 96 |
| Water | <12 | 1 to 9 | 3 to 8 |
| Acetic Acid | <1 | 0.001 to 0.1 | 0.005 to 0.01 |
| Ethyl Acetate | <5 | 0.001 to 4 | 0.01 to 3 |
| Residue | | | |
| Water | 75 to 100 | 80 to 100 | 90 to 100 |
| Ethanol | <0.8 | 0.001 to 0.5 | 0.005 to 0.05 |
| Ethyl Acetate | <1 | 0.001 to 0.5 | 0.005 to 0.2 |
| Acetic Acid | <2 | 0.001 to 0.5 | 0.005 to 0.2 |

Any of the compounds that are carried through the distillation process from the feed or crude reaction product generally remain in the third distillate in amounts of less 0.1 wt. %, based on the total weight of the third distillate composition, e.g., less than 0.05 wt. % or less than 0.02 wt. %. In one embodiment, one or more side streams may remove impurities from any of the columns 207, 208 and/or 209 in the system 200. Preferably at least one side stream is used to remove impurities from the third column 209. The impurities may be purged and/or retained within the system 200.

As shown in FIG. 2, the second distillate is fed via line 220 to fourth column 223, also referred to as the “acetaldehyde removal column.” In fourth column 223 the second distillate is separated into a fourth distillate, which comprises acetaldehyde, in line 224. The fourth distillate preferably is refluxed at a reflux ratio of from 1:20 to 20:1, e.g., from 1:15 to 15:1 or from 1:10 to 10:1, and a portion of the fourth distillate may returned to the reaction zone 201 (not shown). For example, the fourth distillate may be combined with the acetic acid feed, added to the vaporizer 210, or added directly to the reactor 203 (not shown). In another embodiment, the fourth distillate is co-fed with the acetic acid in feed line 205 to vaporizer 210 (not shown). Without being bound by theory, since acetaldehyde may be hydrogenated to form ethanol, the recycling of a stream that contains acetaldehyde to the reaction zone increases the yield of ethanol and decreases byproduct and waste generation. In another embodiment, the acetaldehyde may be collected and utilized, with or without further purification, to make useful products including but not limited to n-butanol, 1,3-butanediol, and/or crotonaldehyde and derivatives (not shown).

The fourth residue of fourth column 223 in line 225 primarily comprises ethyl acetate and water and is highly suitable for use as an ester feed stream. In one preferred embodiment, the acetaldehyde is removed from the second distillate in fourth column 223 such that no detectable amount of acetaldehyde is present in the residue of column 223.

Fourth column 223 is preferably a tray column as described above and preferably operates above atmospheric pressure. In one embodiment, the pressure is from 120 KPa to 5,000 KPa, e.g., from 200 KPa to 4,500 KPa, or from 400 KPa to 3,000 KPa. In a preferred embodiment the fourth column 123 may operate at a pressure that is higher than the pressure of the other columns.

The temperature of the fourth distillate exiting in line 224 from fourth column 223 preferably is from 60° C. to 110° C., e.g., from 70° C. to 100° C. or from 75° C. to 95° C. The temperature of the residue exiting from fourth column 125 preferably is from 70° C. to 115° C., e.g., from 80° C. to 110° C. or from 85° C. to 110° C. Exemplary components of the distillate and residue compositions for fourth column 109 are provided in Table 6 below. It should also be understood that the distillate and residue may also contain other components, not listed, such as components in the feed.

TABLE 6
FOURTH COLUMN

| | Conc. | Conc. | Conc. |
|---|---|---|---|
| | (wt. %) | (wt. %) | (wt. %) |
| Distillate | | | |
| Acetaldehyde | 2 to 80 | 2 to 50 | 5 to 40 |
| Ethyl Acetate | <90 | 30 to 80 | 40 to 75 |
| Ethanol | <30 | 0.001 to 25 | 0.01 to 20 |
| Water | <25 | 0.001 to 20 | 0.01 to 15 |
| Acetal | <1 | <0.05 | <0.001 |
| Residue | | | |
| Ethyl Acetate | 40 to 100 | 50 to 100 | 60 to 100 |
| Ethanol | <40 | 0.001 to 30 | 0 to 15 |
| Water | <25 | 0.001 to 20 | 2 to 15 |
| Acetaldehyde | <1 | 0.001 to 0.5 | Not detectable |
| Acetal | <1 | <0.05 | <0.001 |

In some other embodiments, the second distillate 220 or portion thereof may be returned reactor 203 without passing through the fourth column 223. In some embodiments, it may be advantageous to return a portion of second distillate 220 to reactor 203 when no acetic acid is returned via first residue 216 to reactor 203. Second distillate 220 may also be hydrolyzed or fed to an hydrogenolysis reactor to produce ethanol from ethyl acetate. Additionally, second distillate 220 may be purged from system.

Although one reactor and one flasher are shown in FIG. 2, additional reactors and/or components may be included in various optional embodiments of the present invention. For example, a hydrogenation system may comprise dual reactors, dual flashers, heat exchangers, and pre-heaters.

As shown in FIG. 2, one or more vent streams 226-229 may be withdrawn from the overheads of distillation columns 207, 208, 209, and/or 223. The vent streams may be withdrawn from the non-condensed overhead or from an overhead receiver (not shown). Generally vent streams 226-229 are combined and fed to multi-stage scrubber 230 as shown by line 231. In addition to vent streams 226-229, the inventive ethanol production process may utilize additional vent streams, which may be withdrawn from other units associated with the process, e.g., from reactors, holding tanks, feed tanks, and product tanks, transportation containers, waste water containers, and analyzer exhausts. This listing of potential vent stream sources is not all-inclusive and is not meant to limit the scope of the invention. These vent streams may be fed to the multi-stage scrubbing unit either individually or collectively.

In one embodiment, vent streams 226-229 are fed to a lower portion of a multi-stage scrubbing unit 230, preferably fed to a lower portion of first stage 232 above the exiting bottoms 235. Optionally, there may be independent multi-stage scrubbers 230 for each vent stream 226-229.

In one embodiment, vent streams 226-229, comprise volatile organics and/or entrained ethanol. The volatile organics may comprise acids, esters, aldehydes, ketones, acetals, alcohols having three or more carbon atoms, and mixtures thereof. Suitable volatile organics may include, but are not limited to, acetic acid, ethyl acetate, acetaldehyde, acetal, acetone, ethyl butyrate, ethyl ether, ethyl propionate, isobutyl ethyl ether, isopropanol, isopropyl acetate, methyl acetate, propionic acid, n-butyl acetate, n-propanol, n-propyl acetate, and sec-butyl acetate. Preferably, volatile organics may comprise acetic acid, ethyl acetate, acetaldehyde, acetal, and mixtures thereof, which may be recovered and recycled to the reaction zone 201 and/or separation zone 202. Vent streams 226-229 may further comprise water.

Exemplary component ranges for the combined vent streams are provided in Table 7 below.

TABLE 7
VENT STREAM COMPONENT RANGES

| | Conc. | Conc. | Conc. |
|---|---|---|---|
| | (wt. %) | (wt. %) | (wt. %) |
| Non-condensable gases | | | |
| Nitrogen | 0 to 99.9 | 5 to 99.9 | 10 to 99.9 |
| Others (hydrogen, CO, | 0 to 99.9 | 5 to 99.9 | 10 to 99.9 |
| CO₂, alkanes) | | | |
| Volatile Organics | | | |
| Ethyl Acetate | 0 to 40 | 0.01 to 25 | 0.01 to 10 |
| Acetic Acid | 0 to 40 | 0.01 to 25 | 0.01 to 10 |
| Acetaldehyde | 0 to 40 | 0.01 to 25 | 0.01 to 10 |
| Entrained Ethanol | 0 to 40 | 0.01 to 25 | 0.01 to 10 |
| Water | 0 to 15 | 0.1 to 10 | 0.1 to 5 |

Multi-stage scrubbing unit 230 in FIG. 2, operates in a manner similar as described above in FIG. 1. In the first stage 232, vent streams 226-229 are fed as indicated by input line 231. A first scrubbing solvent 234 is fed to the upper portion of first stage 232. Preferably, the first scrubbing solvent 234 is an acidic solvent, such as acetic acid. First scrubbing solvent 234 may be obtained from acetic acid feed 205 or from first residue 216. Bottoms stream 235 is withdrawn from first stage 232, preferably continuously.

Intermediate stream 236 is withdrawn from an upper portion of first stage 232 and fed to second stage 233. Second scrubbing solvent stream 237 is fed to the upper portion of second stage 233. The second scrubbing solvent may be an aqueous solvent comprising water. In one embodiment, the water may be obtained from first residue 216 and/or third residue 221. As a result of the multiple scrubbing stages, the volatile organics and/or entrained ethanol may be effectively recovered from one or more the vent streams. The remaining scrubbed vent stream 239 is withdrawn from multi-stage scrubber and contains less than 5% of the total amount of volatile organics and/or entrained ethanol that were initially present in the vent stream(s).

Also, in addition to the scrubbed vent stream, each scrubbing stage yields an enriched bottoms or return stream. These enriched streams, in preferred embodiments, may be directed to further processing or may be recycled to the process. For example, in an embodiment where first stage 232 employs an acid solvent, bottoms 235 may be recycled to the reaction zone 201, e.g., to vaporizer 210 or reactor 203. In one embodiment, bottoms stream 235 may be an acetic acid rich stream comprising acetic acid, as well as ethanol, ethyl acetate, and acetaldehyde. Return stream 238 from second stage 233 may be returned to separation zone 202, e.g., to first column 207 or second column 208. In one embodiment, return stream 238 may comprise, in addition to acetic acid, ethanol and/or acetaldehyde.

Multi-stage scrubbing unit 230 and/or the stages thereof may comprise any distillation column capable of separation and/or purification. Multi-stage scrubbing unit 230 preferably comprises a tray column having from 1 to 65 trays, e.g., from 2 to 55 trays, or from 5 to 35 trays. In terms of limits, multi-stage scrubbing unit 230 may comprise less than 65 trays, e.g., less than 55 trays, or less than 35 trays. The trays may be sieve trays, fixed valve trays, movable valve trays, or any other suitable design known in the art. In other embodiments, a packed column may be used. For packed columns, structured packing or random packing may be employed.

The trays or packing, in one embodiment, may be arranged as individual stages in the multi-stage scrubbing unit. In another embodiment, the stages may be arranged in separate scrubbing units that are configured to be in communication with one another.

The temperatures and pressures employed in the multiple-stage scrubbing unit may vary widely and may vary from stage to stage. Generally speaking, the multiple-stage scrubbing unit may be operated at pressures ranging from 0.1 KPa to 500 KPa, e.g., from 1 KPa to 200 KPa or from 1 KPa to 100 KPa. The multiple-stage scrubbing unit may be operated at temperatures ranging from 10° C. to 50° C., e.g., from 20° C. to 45° C. or from 25° C. to 40° C.

The multi-stage scrubbing units shown in FIGS. 1 and 2 show one vessel with multiple stages. In other embodiments, the multi-stage scrubbing unit may be in multiple vessels. FIG. 3 shows multi-stage scrubbing unit 300, which employs two scrubbing stages. FIG. 4 shows multi-stage scrubbing unit 300, which employs three scrubbing stages. Additional scrubbing stages may be added, but generally may not further improve the scrubbing of vent streams. First stage 302 receives vent gas stream(s) 306 from the reaction and/or separation zones as described above in FIGS. 1 and 2, along with first scrubbing solvent stream 308. Preferably, the first scrubbing solvent stream comprises acetic acid. In first stage 302, the scrubbing solvent is supplied to multi-stage scrubbing unit 300 at a point above vent gas stream 306. The first scrubbing solvent scrubs the vent gas to recover volatile organics and/or entrained ethanol. Bottoms stream 307, preferably enriched in acetic acid, exits first stage 302 and is returned to reaction zone.

Intermediate stream 310, preferably in vapor form, exits first stage 302 and is directed to a lower portion of second stage 304. In addition to the contents of intermediate stream 310, second stage 304 also receives second scrubbing solvent stream 312. Preferably, the second scrubbing solvent comprises water. In second stage 304, the scrubbing solvent is supplied at a point above line 310. The second scrubbing solvent scrubs the contents of line 310 to recover volatile organics, entrained ethanol, and or acetic acid (which remains from the first stage) present in intermediate stream 310. Scrubbed vent stream 316 may be purged from system and contains small amounts of volatile organics and/or entrained ethanol, e.g., less volatile organics and/or entrained ethanol than vent stream(s) 306.

Return stream 314, preferably a bottoms streams, exits second stage 304. Return stream 314 may comprise ethanol, water, and acetic acid. In one embodiment, return stream, e.g., enriched water stream 314, may be recycled, e.g., to the separation zone to 1) recover entrained ethanol, 2) further separate impurities from ethanol, and/or may be used to provide process water to separation zone units.

FIG. 4 shows multi-stage scrubbing unit 300, as shown in FIG. 3, with an additional scrubbing stage. Generally, third stage 318 is similar in structure and operation to second stage 304. In the embodiment of FIG. 4, first stage 302 and second stage 304 operate in a manner as described above, except that an additional intermediate stream, i.e. second intermediate stream, 316 is withdrawn from second stage 304 and is fed to third stage 318. In addition to the contents of line 316, third stage 318 also receives third scrubbing solvent stream 320. Preferably, the third scrubbing solvent comprises water and may be provided from the same source as second solvent stream 312. In third stage 318, the scrubbing solvent is supplied at a point above line 316. The third scrubbing solvent scrubs the contents of line 316 to recover volatile organics and/or entrained ethanol that remain after first and second stages 302, 304. Preferably, the water for the third scrubbing solvent has a low impurity level of less than 5 wt. %, e.g. less than 3 wt. % or less than 1 wt. %, where impurities refer to any organic compound in the water. The feed ratio of the third scrubbing solvent to second scrubbing solvent is from 100:1 to 1:100, e.g., from 50:1 to 1:50 or from 10:1 to 1:10. Preferably, the fed ratio of the third scrubbing solvent to second scrubbing solvent is great than 5:1, 10:1 or 50:1.

Second return stream 322 exits the bottom of third stage 318 and preferably comprises ethanol and water. Preferably, second return stream 322 is substantially free of acetic acid. Similar to return stream 314, second return stream 322 may also be returned to the separation zone. Scrubbed vent stream 324 exits the top of third stage 318 and preferably comprises substantially no volatile organics and/or entrained ethanol.

In one embodiment, because of the separation efficiencies achieved therein, the inventive multi-stage scrubbing unit is smaller than a comparable scrubbing unit that utilizes only a single scrubbing stage. In one embodiment, the multiple scrubbing stages are configured in a multi-stage scrubbing unit having a diameter less than 80% of the diameter of a comparable scrubbing unit, e.g., less than 70% or less than 50%. In another embodiment, the multiple scrubbing stages are configured in a multi-stage scrubbing unit having a height less than 80% of the height of a comparable scrubbing unit, e.g., less than 70% or less than 50%. As such, the inventive multi-stage scrubbing unit may be operated at significantly lower energy levels.

Preferably, the multi-stage scrubbing unit is configured such that return stream 322 from third stage 318 is directed to the light ends column where it may be utilized as an extraction agent and/or to recover ethanol. Return stream 314 may be directed to the acid separation column to further separate the recovered volatile organics and/or to recover ethanol. In one embodiment, return stream 322 may be significantly larger than return stream 314, preferably at least twice as large. For example, the flow rate of return stream 322 is at least 100% larger than return stream 314, e.g. at least 500% larger or at least 1000% larger. Generally, the addition of impurity-containing water to the acid column may add to the overall energy load of the acid column when most of the acid is withdraw as the residue. In some embodiments having three scrubbing stages, return stream 314 may be directed to the acid column and return stream 322 may be directed to the light ends column. By directing a smaller stream to the acid column and a larger stream to the light ends column, the energy load of the acid column, beneficially, is reduced. Also, because much of the scrubbing water is returned to the light ends column, the water balance between the product column and the light ends column, advantageously, is maintained. Thus, effective volatile separation and recovery are achieved while 1) the increase in energy load on the acid column is reduced and/or, 2) the water balance between the product column and the light ends column is maintained.

Third distillate 219 may be further purified to form an anhydrous ethanol product stream, i.e., “finished anhydrous ethanol,” using one or more additional separation systems, such as, for example, distillation columns (e.g., a finishing column), membranes, adsorption units, or molecular sieves. Anhydrous ethanol may be suitable for fuel applications.

The final ethanol product produced by the process of the present invention may be taken from the third distillate. The ethanol product may be an industrial grade ethanol comprising from 75 to 96 wt. % ethanol, e.g., from 80 to 96 wt. % or from 85 to 96 wt. % ethanol, based on the total weight of the ethanol product. Exemplary finished ethanol compositional ranges are provided below in Table 8.

TABLE 8
FINISHED ETHANOL COMPOSITIONS

| | Conc. | Conc. | Conc. |
|---|---|---|---|
| Component | (wt. %) | (wt. %) | (wt. %) |
| Ethanol | 75 to 96 | 80 to 96 | 85 to 96 |
| Water | <12 | 1 to 9 | 3 to 8 |
| Acetic Acid | <1 | <0.1 | <0.01 |
| Ethyl Acetate | <2 | <0.5 | <0.05 |
| Acetal | <0.05 | <0.01 | <0.005 |
| Acetone | <0.05 | <0.01 | <0.005 |
| Isopropanol | <0.5 | <0.1 | <0.05 |
| n-propanol | <0.5 | <0.1 | <0.05 |

The finished ethanol composition produced by the embodiments of the present invention may be used in a variety of applications including applications such as fuels, solvents, chemical feedstocks, pharmaceutical products, cleansers, sanitizers, hydrogenation transport or consumption. In fuel applications, the finished ethanol composition may be blended with gasoline for motor vehicles such as automobiles, boats and small piston engine aircrafts. In non-fuel applications, the finished ethanol composition may be used as a solvent for toiletry and cosmetic preparations, detergents, disinfectants, coatings, inks, and pharmaceuticals. The finished ethanol composition may also be used as a processing solvent in manufacturing processes for medicinal products, food preparations, dyes, photochemicals and latex processing.

The finished ethanol composition may also be used as a chemical feedstock to make other chemicals such as vinegar, ethyl acrylate, ethyl acetate, ethylene, glycol ethers, ethylamines, aldehydes, and higher alcohols, especially butanol. In the production of ethyl acetate, the finished ethanol composition may be esterified with acetic acid. In another application, the finished ethanol composition may be dehydrated to produce ethylene. Any known dehydration catalyst can be employed to dehydrate ethanol, such as those described in U.S. Pub. Nos. 2010/0030002 and 2010/0030001, the entire contents and disclosures of which are hereby incorporated by reference. A zeolite catalyst, for example, may be employed as the dehydration catalyst. Preferably, the zeolite has a pore diameter of at least about 0.6 nm, and preferred zeolites include dehydration catalysts selected from the group consisting of mordenites, ZSM-5, a zeolite X and a zeolite Y. Zeolite X is described, for example, in U.S. Pat. No. 2,882,244 and zeolite Yin U.S. Pat. No. 3,130,007, the entireties of which are incorporated herein by reference.

In order that the invention disclosed herein may be more efficiently understood, an example is provided below. The following examples describe the various distillation processes of the present invention.

### Example

A combined vent stream was collected from an ethanol production process in accordance with the invention. The composition of the combined vent stream is shown in Table 9.

TABLE 9
VENT STREAM COMPOSITION

| Component | Wt % |
|---|---|
| Nitrogen | 75.5 |
| Ethyl Acetate | 9.5 |
| Acetic Acid | 5.5 |
| Acetaldehyde | 2.9 |
| Hydrogen | 2.3 |
| Ethanol | 2.2 |
| Water | 2.0 |

The vent stream was directed to a three stage scrubbing unit in accordance with the embodiment shown in FIG. 4. The first stage employed an acetic acid scrubbing solvent. The second and third stages employed a water scrubbing solvent. The scrubbing unit was operated at a top pressure ranging from 0.5 to 2 psig, a bottom pressure ranging from 4 to 6 psig, and at a temperature ranging from 35° C. to 40° C. After scrubbing in the three stage scrubbing unit, the scrubbed vent stream had the composition shown in Table 10.

TABLE 10
EXIT STREAM COMPOSITION

| Component | Wt % |
|---|---|
| Nitrogen | 92.8 |
| Water | 4.4 |
| Hydrogen | 2.8 |
| Acetaldehyde | 0.02 |
| Ethanol | 0.003 |
| Acetic Acid | 0.001 |
| Ethyl Acetate | 0 |

As shown in Table 10, the use of a multi-stage scrubbing unit provides for effective recovery of volatile organics and/or entrained ethanol from the combined vent stream.

While the invention has been described in detail, modifications within the spirit and scope of the invention will be readily apparent to those of skill in the art. In view of the foregoing discussion, relevant knowledge in the art and references discussed above in connection with the Background and Detailed Description, the disclosures of which are all incorporated herein by reference. In addition, it should be understood that aspects of the invention and portions of various embodiments and various features recited below and/or in the appended claims may be combined or interchanged either in whole or in part. In the foregoing descriptions of the various embodiments, those embodiments which refer to another embodiment may be appropriately combined with other embodiments as will be appreciated by one of skill in the art. Furthermore, those of ordinary skill in the art will appreciate that the foregoing description is by way of example only, and is not intended to limit the invention.

We claim:

1. A process for producing ethanol, the process comprising:
(a) hydrogenating an acetic acid feed stream to form a crude ethanol product; and
(b) separating at least a portion of the crude ethanol product to form an ethanol stream and at least one vent stream comprising non-condensable gases and at least one volatile organic;
(c) scrubbing with a first scrubbing solvent at least a portion of the at least one vent stream to form a bottoms stream and an intermediate stream; and
(d) scrubbing with a second scrubbing solvent different from the first scrubbing solvent at least a portion of the intermediate stream to form a scrubbed vent stream.

2. The process of claim 1, wherein the scrubbed vent stream comprises volatile organics in an amount less than 2 wt. %.

3. The process of claim 1, wherein the at least one volatile organic comprises acetic acid, ethyl acetate, acetaldehyde, acetal, and mixtures thereof.

4. The process of claim 1, wherein the at least one vent stream further comprises entrained ethanol.

5. The process of claim 4, wherein the scrubbed vent stream comprises entrained ethanol in an amount less than 2 wt. %.

6. The process of claim 4, wherein the scrubbed vent stream comprises:
up to 99.9 wt. % non-condensable gases;
0.01 to 2 wt. % acetic acid;
0.01 to 2 wt. % ethanol;
0.01 to 2 wt. % acetaldehyde; and
0.0001 to 0.5 wt. % ethyl acetate.

7. The process of claim 1, wherein the scrubbed vent stream comprises less than 5% of the amount of volatile organics in the at least one vent stream.

8. The process of claim 1, wherein the first scrubbing solvent is at a temperature ranging from 10° C. to 50° C. and the second scrubbing solvent is at a temperature ranging from 10° C. to 50° C.

9. The process of claim 1, wherein the first scrubbing solvent comprises acetic acid and the second scrubbing solvent comprises water.

10. The process of claim 1, wherein the bottoms stream comprises acetic acid and ethyl acetate and wherein the bottoms stream is recycled to a hydrogenation reactor.

11. The process of claim 1, wherein step (b) comprises:
separating at least a portion of the crude ethanol product in a first column into a first distillate comprising ethanol, water and ethyl acetate, and a first residue comprising acetic acid;
separating at least a portion of the first distillate in a second column into a second distillate comprising ethyl acetate, and a second residue comprising ethanol and water;
separating at least a portion of the second residue in a third column into a third distillate comprising ethanol, and a third residue comprising water; and
separating at least a portion of the second distillate in an fourth column into a fourth distillate comprising acetaldehyde, and a fourth residue comprising ethyl acetate,
wherein the at least one vent stream is withdrawn from one or more of the first, second, third or fourth columns.

12. The process of claim 11, wherein the second scrubbing solvent used in step (d) comprises at least a portion of the first residue and/or third residue.

13. The process of claim 11, wherein step (d) forms a first return stream comprising acetic acid and wherein the first return stream is recycled to the first column.

14. The process of claim 11, further comprising repeating step (d) with a third scrubbing solvent different from the first scrubbing solvent.

15. The process of claim 14, wherein repeated step (d) forms a second return stream and wherein the second return stream is recycled to the second column.

16. The process of claim 1, wherein the acetic acid in the acetic acid feed stream is formed from methanol and carbon monoxide, wherein each of the methanol, the carbon monoxide, and hydrogen for the hydrogenating step is derived from syngas, and wherein the syngas is derived from a carbon source selected from the group consisting of natural gas, oil, petroleum, coal, biomass, and combinations thereof.

17. A process for producing ethanol, the process comprising:
(a) hydrogenating a first portion of an acetic acid feed stream to form a crude ethanol product; and
(b) separating at least a portion of the crude ethanol product to form an ethanol stream, at least one vent stream comprising non-condensable gases and at least one volatile organic, and a water stream;
(c) scrubbing at least a portion of the at least one vent stream with a second portion of the acetic acid feed stream to form an intermediate stream; and
(d) scrubbing at least a portion of the intermediate stream with at least a portion of the water stream to form a scrubbed vent stream.

18. The process of claim 17, wherein the water stream comprises less than 5 wt. % impurities.

19. The process of claim 17, wherein the scrubbed vent stream comprises volatile organics in an amount less than 2 wt. %.

20. The process of claim 17, wherein the at least one vent stream further comprises entrained ethanol.

21. A process for producing ethanol, the process comprising:
(a) hydrogenating an acetic acid feed stream to form a crude ethanol product; and
(b) separating at least a portion of the crude ethanol product to form an ethanol stream and at least one vent stream comprising non-condensable gases and at least one volatile organic;
(c) scrubbing with a first scrubbing solvent at least a portion of the at least one vent stream to form a bottoms and a first intermediate stream;
(d) scrubbing with a second scrubbing solvent different from the first scrubbing solvent at least a portion of the first intermediate stream to form a second intermediate stream; and
(e) scrubbing with a third scrubbing solvent different from the first scrubbing solvent at least a portion of the second intermediate stream to form a scrubbed vent stream.

22. The process of claim 21, wherein the scrubbed vent stream comprises volatile organics in an amount of less than 2 wt. %.

23. The process of claim 21, wherein the at least one vent stream further comprises entrained ethanol.

24. The process of claim 21, wherein the first scrubbing solvent comprises acetic acid and the second and third scrubbing solvents comprise water.

25. A process for producing ethanol, the process comprising:
(a) providing a stream comprising non-condensable gases and at least one volatile organic;
(b) scrubbing with a first scrubbing solvent at least a portion of the at least one vent stream to form a bottoms stream and an intermediate stream; and
(c) scrubbing with a second scrubbing solvent different from the first scrubbing solvent at least a portion of the intermediate stream to form a scrubbed vent stream.
